(19) United States
(12) Patent Application Publication
(10) Pub. No.: US 2014/0276889 A1
(43) Pub. Date: Sep. 18, 2014

(54) PORTABLE SURGICAL GUIDE WITH LASER, ABDUCTION AND ANTEVERSION MEASURING SYSTEM AND METHOD OF USING SAME

(71) Applicants: William C. HEAD, Frisco, TX (US); Steven DeLeon, Austin, TX (US); Bart Dalton, Frisco, TX (US)

(72) Inventors: William C. HEAD, Frisco, TX (US); Steven DeLeon, Austin, TX (US); Bart Dalton, Frisco, TX (US)

(21) Appl. No.: 14/213,010

(22) Filed: Mar. 14, 2014

Related U.S. Application Data
(60) Provisional application No. 61/798,678, filed on Mar. 15, 2013.

(51) Int. Cl. A61F 2/46

(52) U.S. Cl. CPC A61F 2/4657
USPC 606/102

(57) ABSTRACT

Methods, systems and devices for properly positioning and aligning a prosthetic socket and/or prosthetic ball into host bone structure of a patient using one or more guide shown. Guides implemented according to embodiments may utilize in combination digital IC inclinometer and the laser surgical system with calibrated, reflecting dome.

### CROSS-REFERENCE TO RELATED APPLICATIONS

This application claims priority to co-pending U.S. Provisional Patent Application No. 61/798,678, entitled “PORTABLE SURGICAL GUIDE WITH LASER, ABDUCTION AND ANTEVERSION MEASURING SYSTEM AND METHOD OF USING SAME,” filed Mar. 15, 2013, the disclosure of which is hereby incorporated herein by reference.

This application is related to U.S. patent application Ser. No. 12/371,308, to William C. Head (the '308) now U.S. Pat. No. 8,469,962, filed Feb. 13, 2009, the disclosure of which is hereby incorporated herein by reference.

This application is further related to U.S. patent application Ser. No. 12/692,449, to William C. Head (the '449) now U.S. Pat. No. 8,454,619, filed Jan. 22, 2010, the disclosure of which is hereby incorporated herein by reference.

The '449 application is a continuation-in-part of '308 application, which itself is a continuation-in-part of the U.S. application Ser. No. 12/332,109 entitled Prosthetic Socket Alignment, filed Dec. 10, 2008, the disclosure of which is hereby incorporated by reference for all purposes.

### TECHNICAL FIELD

The present invention relates to a portable surgical positioning apparatus and system and, in particular, a Portable Surgical Guide with Laser (PSG) for orienting surgical instruments, implants and prosthetics, and methods of using same. The device is configured as an instrument of small size, low economic cost due to the use of standardized components and their structural simplicity.

### BACKGROUND OF THE INVENTION

The proper angle of cup abduction and anteversion during placement of a prosthetic device is critical for hip stability and minimizes acetabular and femoral component wear.

Though medical practitioners have been performing hip replacement surgeries for more than half a century, improper angle of cup abduction and anteversion is a common problem causing: 1) a dislocation of the prosthetic ball from the prosthetic socket (i.e., the prosthetic ball comes out of the prosthetic socket) and 2) an excessive wear during use of the prosthetic implant depending, to some extent, on the materials used to make the prosthetic ball and socket. For example, excessive wear occurs when the prosthetic ball rubs excessively on one edge of the socket. This is known as edge loading. Wear debris, such as formed by edge loading in the joint, can cause major complications such as inflammation and loosening of the prosthetic components. Moreover, when the prosthetic components wear out or loosen, they have to be replaced in another hip replacement surgery.

Because of the problems associated with improperly aligned prosthetic sockets and balls (due to improper angle of cup abduction and anteversion during placement of a prosthetic device), medical practitioners generally make every effort to try and properly align these devices during surgery, starting with an effective preparation of a bone cavity by a reamer. Most medical practitioners rely on their experience to view the bone cavity and manually place the prosthetic socket in the proper position. For example, the proper alignment of the hemispherical socket in the acetabula (the cup-shaped cavity in the pelvis into which the ball-shaped head of the femur fits) is typically attained when it is about 40° to 45° of abduction. Additionally, when the prosthetic socket is properly aligned in the acetabula, the open area of the socket typically should be about 10° to 20° of anteversion, i.e., facing forward. It should be noted that though the abduction angle is typically about 40° to 45° and the anteversion angle is typically about 10° to 20°, variations outside these ranges are possible and the embodiments of the invention disclosed herein may be used in instances outside of the typical ranges.

Particularly because medical practitioners are now performing surgeries with smaller incisions than have traditionally been used, it is not uncommon that manual fitting of a prosthetic socket during hip surgery results in the prosthetic socket being placed at an angle of 50° to 60° and even facing slightly backward (retroversion). Such improper alignment generally results in dislocations of the hip and excessive wear of the prosthetic ball and socket after surgery as mentioned above. To improve upon manual alignment of prosthetic sockets, medical practitioners have tried to use positioning devices based on x-ray, fluoroscopy, MRI and other electronic technology, including variety of tilt sensors. Such devices pose portability issues, often are very expensive and complicated to operate, require additional trained personnel to operate and generally proved ineffective. Despite these technologies, improper alignments of prosthetic balls and sockets persist and these improper alignments in turn cause complications such as dislocations and excessive wear.

### SUMMARY OF THE INVENTION

In accordance with the present invention, an Abduction and Anteversion Measuring System (AAMS) and methods are provided for properly positioning and aligning a prosthetic socket in a bone cavity of a patient.

In accordance with an exemplary embodiment of the present invention, an AAMS comprising a Portable Surgical Guide with Laser (PSG) and a calibrated, reflective dome is provided. The AAMS will guide the surgeon to properly position the acetabular component in two planes (sagittal and coronal) to secure proper angles of abduction and anteversion.

The PSG is a hand-held, portable device comprising an enclosed unit with a digital inclinometer and a laser. According to one embodiment of the present invention, surgeons read the abduction angle with an integrated circuit system (ICS) and mark the anteversion position with a laser guide system (LGS), said LSG comprising the reflective, calibrated dome and the laser. According yet to another embodiment of the present invention (when the patient is in a supine position), surgeons mark the abduction angle with the LGS and read the anteversion angle with the ICS. The PSG is configured to orient off a calibrated, reflective dome, said dome attached to a guiding piece such as a Stainmann pin guide, said pin fixedly inserted into a pelvis.

The PSG may be reversibly attached to various prosthetic alignment devices such as a hip reamer or an acetabular driver. The system may be used in conjunction with straight or offset reamer handles, trialing handles and impaction devices. The exemplary system utilizing the Offset Reamer Driver (ORD), which easily introduces acetabular reamers to the hip joint may be of Symmetry Medical, Inc.® make.

The present invention provides many important technical advantages. One important technical advantage of the present invention is a system and method of properly orienting prosthetic socket by utilizing a hand-held portable and disposable device of remarkable precision in establishing abduction/anteversion angle by utilizing a combination of techniques. Not only will this disposable system assist surgeons in the proper placement of the acetabular component in the abduction plane, it will also guide the surgeon for anteversion placement.

The embodiments of the present disclosure alleviate to a great extend the disadvantages of using a complex to operate and very expensive devices to properly measure an abduction and an anteversion angles of a prosthetic cup.

Those skilled in the art will further appreciate the advantages and superior features of the invention together with other important aspects thereof on reading the detailed description that follows in conjunction with the drawings.

### BRIEF DESCRIPTION OF THE DRAWINGS

For a more complete understanding of the present invention, reference is now made to the following descriptions taken in conjunction with the accompanying drawings, in which:

FIG. 1 shows a perspective-front view of the AAMS in accordance with an exemplary embodiment of the present invention.

FIG. 2 shows a perspective-bottom view of the PSG in accordance with an exemplary embodiment of the present invention.

FIG. 3 shows a cross-sectional view of the PSG in accordance with an exemplary embodiment of the present invention.

FIG. 4 shows an isometric view of a calibrated, reflective dome in accordance with an exemplary embodiment of the present invention.

FIG. 5 shows a perspective-bottom view of a calibrated, reflective dome in accordance with an exemplary embodiment of the present invention.

FIG. 6A is a perspective view of a reamer with the PSG in a desired position of abduction of the left acetabulum in accordance with an exemplary embodiment of the present invention.

FIG. 6B is a view from above with Steinmann pin at the 12 o'clock position and view of a reamer with the PSG in a desired position of anteversion in accordance with an exemplary embodiment of the present invention.

FIGS. 7 (lateral view) and 8 (view from above) show a patient disposed laterally (on his left side) for performing an implant procedure using AAMS of angles relative to imaginary planes through a patient's body in accordance with an exemplary embodiment of the present invention.

FIG. 9 shows a calibrated dome placed over Steinmann pin and the PSG attached to a reamer in accordance with an exemplary embodiment of the present invention.

FIG. 10 is a perspective view of the AAMS showing operator holding the PSG in accordance with an exemplary embodiment of the present invention.

FIG. 11 shows a process to properly align a prosthetic ball in accordance with an embodiment of the present invention.

### DETAILED DESCRIPTION OF THE INVENTION

As used herein, the following terms should be understood to have the indicated meanings:

When an item is introduced by “a” or “an,” it should be understood to mean one or more of that item.

“Battery” means a portable source of electrical power.

In FIG. 1, the Abduction and Anteversion Measuring System (AAMS) 10 comprises a hand-held, Portable Surgical Guide with Laser (PSG) 1 powered by battery which is fitted into a battery cartridge 2 (shown closed), and a calibrated, reflective dome 3.

An exemplary PSG 1 of a preferred embodiment of the invention is shown in FIGS. 1, 2 and 3. As shown in FIGS. 1 and 2, the exemplary PSG 1 comprises a casing 1A extending downwardly to form a waive-shaped ergonomic handle 1B.

In FIGS. 2 and 3, an exemplary PSG 1 has been proposed as a unitary guiding device with laser 4. The exemplary PSG 1 includes motion sensitive electronic circuitry (an IC inclinometer or digital inclinometer 8) to measure an angle of inclination (here, either an abduction or an anteversion angle, depending on the patient's position) up to decimal point accuracy and displaying the angle on a display 5. The IC inclinometer is housed in casing 1A and activated by actuation means controlled by On/Off button 6, and re-calibrated by actuation of Alt/Zero button 7. The laser source component 4 reads off the dome 3 and hence provides a reading of the anteversion angle or an abduction angle, depending on the patient's position.

In FIG. 3, an exemplary cross-section of PSG 1 is shown and comprises an enclosure front 1C and an enclosure back 1D that when combined together constitute a casing 1A with handle 1B extending downwardly, the casing 1A enclosing integrated circuitry of inclinometer with LCD (IC inclinometer) 8 for measuring an angle of inclination (here, an abduction or an anteversion angle) and a digital display 5 for displaying the measurement depending on the step accuracy for example 01, 05 or 0.01 degree steps. The circuitry of the PSG 1 is known in the art and will not be elaborated in detail. It utilizes a micro-electromechanical motion sensor arrangement which responds to movements of the operating surgeon and which is encapsulated within solid state integrated signal processing means. An example of such commercially available digital component that may be utilized is Digi-Pas® DWL-80E sold by globalindustruial.com.

Unlike known digital guides, the PSG 1 further includes a laser 4 which is actuated by a laser switch 9 (On/Off). An example of such commercially available laser switch that may be utilized with the PSG 1 is a nylon/zinc plate steel switch laser available through www.uxcell.com or http://www.taydaelectronics.com/. The exemplary switch utilized is Model SS12F48, Type: 1 Pole 2 Throw, 2 Position. Continuing with FIG. 3, laser 4 is disposed within laser mount 11. The laser mount 11 is fitted into the volume space 12 in the upper portion of casing 1A of the exemplary PSG 1. The digital and the laser component of the PSG 1 are powered by a battery enclosed in a battery cartridge 2. The battery cartridge 2 is configured to fit into the battery slot housing 13. The laser switch 9 is electrically connected to the battery solder joints of the IC inclinometer 8 by a short switch wire (not shown). Hence, both digital and laser component of the PSG 1 utilize the same source power.

According to one embodiment of the present invention, the suitable batteries that maybe used are: coin cell Lithium, battery size 2023, voltage 3 diameter ⅘ inches for use with medical devices per ANSI standards (Cross reference: BR 2023; CR 2032; DL 2032; ECR2032 and KECR 2032-1).

According to one embodiment of the present invention, a commercially utilized laser source component 4 may be INSTAPARK® DRM104-DQ01. The up or down trajectory of the light 14 beaming from the laser source component 4 is manually controlled by the operator interfacing a knob indicating laser 15, said knob connected with a laser mount 11 by a laser spacer component 16. An exemplary knob indicating laser may be phenolic, while the spacer may be made of nylon and both commercially available through MCMASTER-CARR®.

In one of the described embodiment, the PSG 1 is made of thermoplastic materials such as ABS (Acrylonitrile/Butadiene/Styrene), including components such as battery cartridge, laser mount, the IC actuating button (on/off), and the re-calibration button, but other material, such as metal, is envisaged. Also, although preferred, it is envisaged that the laser 4 need not be integrally formed with the PSG 1. If formed separately, then the PSG 1 may be assembled with the laser 4 using screws or other suitable attachment means.

According to one embodiment of the present invention the AAMS comprises a calibrated, reflective dome. FIGS. 4 and 5 show an exemplary dome 3 having an orifice 17 in a bottom, center portion of the base 3A of dome 3. Dome 3 maybe calibrated every 10 degrees or (if suitable) every 15 or 30 degrees depending on the size of the utilized dome, and made of reflective ABS or stainless steel to allow the reading of the anteversion angle reflection 18 of the leaser's beaming light 14 on the dome 3. The orifice 17 of dome 3 is shaped to receive the Steinmann pin guide 19 as shown in FIGS. 6 and 9.

The friction created between the dome's center hole 17 and the Steinmann pin allows the dome 3 to be permanently positioned and held in one place during the entire procedure. The dome is positioned (aligned) over the pin such that the imaginary plane going through the zero degrees mark on the dome 3 (such imaginary plane perpendicular to the dome base 3A) is parallel to the coronal plane, an imaginary plane dividing patient's body to front/back 23 as shown in FIG. 8. In combination, the dome 3 and the Steinmann pin 19 constitute a reference guide utilized throughout the entire process of the acetabular placement.

According to one embodiment of the present disclosure, surgeons read the abduction angle with an integrated circuit system (digital IC) and mark the anteversion position utilizing a laser guide system (LGS) as shown in FIGS. 6A and 6B, respectively if the patient is disposed on his side (positioned laterally). However, if the patient is in supine position (not shown), surgeons mark the abduction angle utilizing the LGS and the anteversion angle using digital IC.

In FIG. 6A, the PSG display 5 indicates the angle of abduction from the patient's mid-line X. The ideal abduction cup position is between 40 and 45 degrees. In FIG. 6B, the dome reflects between 10 and 20 degrees of anteversion angle. Surgeons achieve this accuracy and precision with a simple set up prior to beginning surgery (such as positioning devices) and attention to detail during the surgical procedure, as described below.

As shown in FIGS. 7 and 8, the table is positioned parallel to the floor, with the patient in a secure lateral position. As shown in FIG. 7, the abduction angle of a reamer/driver 21 is the angle between the reamer/driver 21 and sagittal plane 22 (an imaginary plane dividing the body into left and right portions). The anteversion angle is the angle between coronal plane 23 (an imaginary plane dividing the body into anterior and posterior portions) and the reamer/driver 21 as shown in FIG. 8. It should be noted that coronal plane 23 and sagittal plane 22 are perpendicular to each other. A cross-table AP x-ray of the pelvis is taken by apparatus 20 to identify position of the pelvis in relationship to the table.

In FIG. 7, the PSG 1 is placed on the table and may be re-zeroed/recalibrated to accommodate operating room tables where the table is tilted (e.g., the table is not parallel with the floor). In one embodiment of the present invention, the PSG 1 is optimized for a patient in the lateral position; however, the PSG 1 may also be used with a patient in a supine position (not shown).

The table position should be verified prior to prepping the patient for a surgery and, once again, the table position should be checked for alignment after the patient has been prepped and draped. The commercially available level could be used along with the PSG 1 placed on the operating table, together constituting the positioning devices. Other positioning devices, such as earlier mentioned in this specification, may be utilized. An example of such positioning device is an x-ray.

As shown in FIG. 9, once the surgery begins, a Steinmann pin guide 19 is placed at the 12 o'clock position above the acetabulum and, more particularly, 1-2 cm above the acetabular rim. The pin 19 has one-inch stop 24 to prevent drilling too deeply into the pelvis.

The calibrated dome 3 is then placed over the Steinmann pin 19 as shown in FIGS. 6, 9 and 10 and aligned such that 0 degrees plane (going through the 0 degrees mark on the dome 3) is perpendicular to the dome's base 3A and parallel to the coronal plane (when the patient is laterally positioned). Should the pelvis move, this point of reference remains constant during the entire operation.

As shown in FIG. 9, once the Steinmann pin 19 and calibrated dome 3 are in place, the acetabular reamer 25 can be properly positioned in both the abduction and anteversion planes using the PSG 1. The starting point should be zero abduction 25-1. First, holding the PSG 1 directly over the reamer, the operator moves the reamer to the desired abduction angle 25-2. Then, using the LGS of the PSG 1, the operator selects the proper angle of anteversion for reaming in both planes.

The PSG 1 is hand-held over the reamer sleeve 25 during this phase of the procedure as shown in FIG. 10. Once the acetabular preparation is complete, the PSG 1 is securely attached to the acetabular driver with a C-clamp for cup implantation (not shown). An attachment means comprising a bracket or a C-clamp (not shown) for releasably attaching the PSG 1 to another article is utilized. According to one embodiment of the present invention, the bracket is made from plastic and integrally formed with the housing 1A and handle 1B of the PSG 1. Each of the two clamping arms may be accurate in shape with an abutment surface for clamping to the article (not shown). In this embodiment, the clamp is specifically adapted for attaching to a rigid body of a reamer.

Exemplary embodiments include a method of achieving proper anteversion and abduction angles of the cup by using a portable system described herein.

FIG. 11 shows process 100, that properly fits a prosthetic socket within the bone cavity of a patient. Prior to surgical incision, it is recommended that a carpenter's level and/or the PSG 1 (hereinafter, the positioning devices) are utilized to ensure that the table is parallel to the floor.

In process 101, using a skin marker to outline the iliac crest, the anterior superior iliac spine 26 as shown in FIG. 8 is identified. In process 102, a cross-table AP x-ray 20 is utilized to demonstrate the position of the pelvis on the operating table. The angle α of the pelvis, which usually falls into a slight adducted position should be then measured as shown in FIG. 7. The acetabulum angle in relationship to the table is calculated and factored into the abduction angle for the cup. For example with a 10-degree angulation of the pelvis and a 40-degree desired cup position, the PSG 1 should be set at 30 degrees.

According to one embodiment of the present invention, the patient is secured in a lateral position and prepared and draped in a conventional manner. The operator than opens the sterile pack comprising the PSG with battery cartridge, a Steinmann pin guide, a calibrated dome, and a clamp to attach the unit to the acetabular driver. A battery cartridge 2 is inserted into the battery housing slot 13 of PSG 1. The batteries in the cartridge should be facing away from the display side as shown in FIG. 3 and inserted straight into the PSG (e.g., do not tilt up or down). The battery cartridge will snap into place and cannot be removed.

In process 103, the PSG 1 is placed flat on the operating table and the integrated circuit 8 is turned on by pressing the On/Off button 6. With the device flat on the table, the display should read 0.0 to 0.5 to indicate that the table is horizontal to the floor. The PSG's accuracy is +/−0.5 degrees on a measurement range of +/−90 degrees. This has been predetermined by the manufacturer on a precisely level surface. Should the device read above 0.5 while resting on the operating table the Alt/Zero button 7 should be engaged until the read-out shows 0.0 to 0.5.

The surgical exposure may begin by dislocating the hip and removing the femoral head. In process 104, the Steinmann pin 19 is then placed at the 12 o'clock position above the acetabulum perpendicular to the operating table. In process 105, the calibrated dome 3 is then placed over the Steinmann pin 19. The calibrated dome 3 is marked so to show increments of 10 degrees or 30 degrees depending on the size of the dome.

When acetabular preparation has begun, the PSG 1 is held over the reamer's sleeve 25 as shown in FIG. 10. The starting point should be at 0 degrees, directly in line with the laser portion of the PSG and the Steinman pin 19. In process 106, the reamer 25 is positioned in the desired abduction position and then moved into a desired anteversion in process 107. The operator proceeds with acetabular preparation in two planes (abduction and anteversion). The final reaming should be at the precise angles for cup implantation. In process 108, when the cup is to be inserted, the PSG 1 is attached to the shaft of the driver (not shown) and impacted in the correct abduction and anteversion angles.

According to embodiments of the present disclosure, the surgeon may ream the acetabulum, trial, and implant the acetabular cup while having the ability to check the angle of abduction throughout the procedure utilizing different handles such as grasp reamer handle, trialing or impaction handle with the PSG 1 resting on top of handle shaft.

To orient the cup position for anteversion, the handle 1B of PSG 1 is aligned with the long mechanical axis of the patient and with an abduction angle at approximately 40 to 45 degrees.

When dealing with an obese patient, there is a tendency for the pelvis to fall into downward position from a lateral approach. It is recommended that in such patients the abduction angle should be 35 degrees in order to accommodate that particular scenario.

It should be noted that the methods, devices and systems described herein are applicable to surgeries at different locations of the body. For example, the procedures described herein may be used in surgeries such as hip replacement surgery, shoulder replacement surgery and knee replacement surgery. Further, the methods, devices and systems include surgeries on humans and surgeries performed in the field of veterinary medicine.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

What is claimed is:

1. A method for properly orienting a prosthetic socket in a bone cavity of a patient, said method comprising:
placing said patient in a lateral position; and
preparing said bone cavity for receiving said prosthetic socket using a reamer, wherein an Abduction and Anteversion Measuring System (AAMS) is used to guide an orientation of said reamer during said preparation to provide a prepared bone cavity facilitating said properly orienting said prosthetic socket.

2. The method of claim 1, wherein said AAMS comprises a Portable Surgical Guide with Laser (PSG).

3. The method of claim 1, wherein said AAMS further comprises a calibrated dome.

4. The method of claim 3, wherein said dome is made of reflective material.

5. The method of claim 1, wherein said AAMS further comprises a Steinmann pin guide placed into a bone, said bone being a bone in which said bone cavity is disposed.

6. The method of claim 4, wherein said dome is permanently disposed on said Stainmann pin guide.

7. The method of claim 2, wherein said PSG is disposed on said reamer.

8. The method of claim 1, further comprising:
establishing a reference guide providing a baseline reference with respect to the patient after said patient is placed in said lateral position but before said bone cavity is prepared for receiving said prosthetic socket.

9. The method of claim 8, wherein said establishing said reference guide is performed prior to any surgical steps for implanting said prosthetic socket.

10. The method of claim 9, wherein said reference guide comprises said Steinmann pin disposed in said patient in a specific orientation and said calibrated dome, said dome disposed on said Stainmann pin guide, wherein said reference guide is referenced to make a measurement using said specific position as a reference during performance of a procedure to orient said prosthetic socket.

11. The method of claim 1, wherein said placing said patient in said lateral position comprises:
engaging a plurality of positioning devices to ensure proper aligning of an implanted prosthetic.

12. An Abduction and Anteversion Measuring System (AAMS), said system comprising:
a hand-held Portable Surgical Guide with Laser (PSG); and
a calibrated dome, said dome disposed in a Steinmann pin guide.

13. The system of claim 12, further comprising an attachment means for releasably attaching the PGS to another article.

14. The system of claim 12, wherein said PSG comprises an integrated circuit inclinometer and a digital display for displaying an abduction angle.

15. The system of claim 12, wherein said PSG comprises an integrated circuit inclinometer and a digital display for displaying an anteversion angle.

16. The system of claim 12, wherein said PSG further comprises a Laser Guiding System (LGS), said LGS further comprising:
a laser source, said laser source being functionally connected to a knob indicating laser, wherein said knob indicating laser is configured to change a trajectory of a light beaming from said laser source, said laser source being controlled by a laser switch.

17. The system of claim 16, further comprising a laser spacer, said laser spacer connecting said laser source with said knob indicating laser.

18. The system of claim 16, said system configured to mark an anteversion angle using said LGS and said reflective dome.

19. The system of claim 16, said system configured to mark an abduction angle using said LGS and said reflective dome.

20. The system of claim 17 wherein said inclinometer's integrated circuit and said laser source of said PSG are utilizing one battery source.
